(19) United States
(12) Patent Application Publication
(10) Pub. No.: US 2006/0135838 A1
(43) Pub. Date: Jun. 22, 2006

(54) Catalyst and method for converting low molecular weight paraffinic hydrocarbons into alkenes

(76) Inventors: Ebrahim Bagherzadeh, Sugarland, TX (US); Abbas Hassan, Sugarland, TX (US); Rayford G. Anthony, College Station, TX (US); Xianchun Wu, College Station, TX (US)

Correspondence Address: Thomas L. Adams, Esq., 120 Eagle Rock Avenue, P.O. Box 340, East Hanover, NJ, 07936, US

(21) Appl. No.: 11/298,160

(22) Filed: Dec. 9, 2005

Related U.S. Application Data
(60) Provisional application No. 60/634,767, filed on Dec. 9, 2004.

(51) Int. Cl. C07C 5/333; C01F 17/00

(52) U.S. Cl. 585/660; 423/263; 423/594.16

(57) ABSTRACT

A process and catalyst for the partial oxidation of low molecular weight paraffinic hydrocarbons, such as methane, ethane, propane, naphtha, and natural gas condensates to form alkenes, such as ethylene, propylene and other valuable by-products. The process involves contacting the low molecular weight paraffinic hydrocarbon with the catalyst in the presence of oxygen or air and optionally steam. The catalyst has a perovskite-type crystalline structure, and lends itself to fixed and fluidized bed reactor configurations. The conversion process is less costly than conventional processes due to low pressure operation, the use of air and steam as a source of oxygen, and lower operating temperatures resulting in less coking, downtime, and reduced cost for materials of construction. Catalyst activity is extended and reactor downtime for catalyst regeneration is minimized by addition of chlorides and/or amines.

### CROSS-REFERENCES TO RELATED APPLICATIONS

This application claims the benefit of U.S. Provisional Patent Application Ser. No. 60/634,767, filed 9 Dec. 2004, the contents of which are hereby incorporated by reference herein in their entirety.

### FIELD OF THE INVENTION

This invention relates to the conversion of low molecular weight paraffinic hydrocarbons into alkenes, especially useful in the production of ethylene from ethane and/or methane through the use of a novel catalyst. Catalyst activity and longevity is enhanced through novel reactor configuration and additive feeds.

### BACKGROUND OF THE INVENTION

Alkenes are unsaturated hydrocarbons that contain one or more carbon-carbon double bonds and include ethylene, propylene, butylenes, butadiene and other alkenes, which are some of the key hydrocarbons used in the petrochemical industries. These hydrocarbons are the primary building blocks in the production of such products as polyethylenes such as low density polyethylene (“LDPE”), high density polyethylene (“HDPE”), linear low density polyethylene (“LLDPE”); polypropylene, polyvinyl chloride (“PVC”), ethylene glycol, and rubbers such as SBR/PBR (styrene butadiene rubber/polybutadiene rubber).

Paraffinic hydrocarbons, also called alkanes, and for the purposes of the present specification, are considered to include any of the saturated hydrocarbons having the general formula CₙH₂ₙ₊₂, where C represents a carbon atom, H represents a hydrogen atom, and n is an integer. The paraffins are major constituents of natural gas and petroleum. Paraffins comprising fewer than 5 carbon atoms per molecule are usually gaseous at room temperature, while those comprising between 5 to 15 carbon atoms are usually liquids at room temperature (Encyclopedia Britannica, 2004). When n is between 22 and 27 the hydrocarbon is solid at room temperature, and is usually referred to as paraffin. The simplest paraffinic hydrocarbon is methane (CH₄) followed by (in terms of increasing number of carbons) ethane, propane, butane and higher aliphatic hydrocarbons.

Ethylene is typically obtained from the non-catalytic thermal cracking of saturated hydrocarbons such as ethane and propane, and alternatively from the thermal or steam cracking of heavier liquids such as naphtha and gas oil. Steam cracking produces a variety of other products, including diolefins and acetylene. The latter are costly to separate from the ethylene, and this is usually done by extractive distillation and/or selective hydrogenation of the acetylene back to ethylene. Thermal cracking processes for olefin production are highly endothermic. Accordingly, these processes require the construction and maintenance of large, capital intensive and complex cracking furnaces to supply the heat for this energy intensive process. Thermal cracking also has the tendency to form coke on the reactor, and this process has to be periodically shutdown for the removal of built-up coke (“de-coking”).

An alternative is to catalytically crack paraffinic hydrocarbons in the presence of oxygen to form mono-olefins, that is, the autothermal partial oxidation of paraffinic hydrocarbons to olefins. The term “partial oxidation” implies that the paraffinic hydrocarbon is not substantially oxidized to carbon monoxide and carbon dioxide, but rather, partial oxidation comprises one or both processes of oxidative dehydrogenation and cracking to form primarily olefins. Under these autothermal process conditions, no external heat source is required. However, substantial amounts of carbon oxides are usually formed, and the selectivity to produce olefins has been low compared to thermal cracking. U.S. Pat. No. 6,566,573 (Bharadwaj et al.) describes such a process but deficiencies involving catalyst life and costly equipment requirements exist.

The present inventors have discovered that certain perovskite based catalysts are effective in the direct conversion of paraffinic hydrocarbons to alkenes and higher hydrocarbons with selectivity for the production of ethylene and other olefins.

Perovskites are a well known class of compounds. U.S. Pat. No. 4,863,971 describes perovskite catalysts as “crystalline, mixed metal oxides having the general empirical formula ABO₃ and containing substantially equal numbers of metal cations at the A and B sites in the perovskite crystal lattice structure.”

The term “perovskite” as used herein is intended to describe mixed metal oxides having the ideal and non-ideal perovskite crystalline structure. The ideal perovskite structure is cubic; however, few compounds have this ideal structure. While a more complete description of the perovskite structure can be found in Structural Inorganic Chemistry, A. F. Wells, 3rd Edition, Clarendon Press, Oxford, U.K., 1962, pages 494 to 499, it should be noted that cation A may comprise more than one metal and cation B may comprise more than one metal. In general, the algebraic sum of the ionic charges of the two or more metals (cations) of the perovskite equals 6. The ideal perovskite structure has also been discussed by Itoh, Mitsuru, Proceedings of the first Symposium on Atomic-Scale Surfaces and Interfaces Dynamics, Mar. 13-14, 1997, Tokyo, Japan.

The preparation of perovskite compounds is known in the art. Procedures for preparing perovskite compounds are disclosed in Structure, Properties and Preparation of Perovskite Type Compounds by Francis Galasso, Pergamon Press, Oxford (U.K.), 1969, and in U.S. Pat. Nos. 4,126,580 and 4,312,955, the contents of which are incorporated herein by reference. Embodiments of the present invention deviate from this ideal ABO₃ structure described by Itoh et al. and have been found to be unexpectedly efficient as an oxidative coupling catalyst.

The stability of the structure of the perovskite-type oxides is evaluated using what is known to those skilled in the art as a tolerance factor. Tolerance factor (“t”) is defined in Proceedings of the First Symposium on Atomic-scale Surface and Interface Dynamics, Mar. 13-14, 1997, Tokyo, Japan.
t=(rₐ+rₒ)/(√2 (r_{b}+rₒ))
where in the crystal structure, rₐ and r_{b} are the ionic radii of cation species a and b, respectively, and rₒ is the ionic radius of the anion species.

Data for the atomic radii used to calculate the tolerance factor of the catalyst embodiments of the present invention were from Lange's Handbook Of Chemistry, J. A. Dean, (ed.), 15^{th} edition, McGraw-Hill, 1999.

This tolerance factor actually determines the properties of perovskite-type oxides. Using the ionic radii for various metal ions, t values can be calculated for real and theoretical perovskite-type oxides. For the purpose of the present invention it has been discovered that a value of ‘t’ ranging from about 0.8 to about t=1.1 provides the best perovskite catalyst structure for conversion of paraffinic hydrocarbons to alkenes. This results in formation of an ideal, or close to ideal cubic shape of the crystal. The existence of this perovskite structure can be confirmed by X-ray diffraction data.

As used herein, the terms “about” or “approximately”, when preceding a numerical value, are intended to have their usual meaning, and this also includes the range of normal measurement variations that is customary with laboratory instruments that are commonly used in the field of endeavor (for example only, and not intended to be limited to, weight, temperature or pressure measuring devices).

The present inventors have discovered that ideal or near ideal perovskite structures can be readily produced through proper selection of raw metal salts and oxides as well as use of a novel sol-gel technique comprising the use of an organic acid to form an organo-metallic compound followed by gel formation and calcination.

The resulting perovskite-containing composition may be combined with conventional supports such as silica, alumina, silica-alumina, silica, zirconia, other inorganic oxides, carbon, etc., to form composite catalysts.

Embodiments of the present invention involve the use of a perovskite catalyst and specific process conditions to convert low molecular weight paraffins, including methane, into more functional alkenes containing one or more double bonds. Methane and, to a lesser extent, ethane are major low molecular weight alkanes found as major components of most natural gas fields around the globe. Converting methane into alkenes, either ethylene or higher carbon number compounds, allows for reactions to create yet higher carbon number materials (generally having greater than six carbon atoms that are liquids and/or solids at ambient conditions, thus reducing some of the drawbacks connected with methane transportation from remote areas.

Ethane conversion to ethylene and other alkenes is also another important chemical reaction that today involves mainly the use of steam crackers.

Steam cracking of ethane is a widely used technology that utilizes mainly heat and no catalyst to dehydrogenate ethane to ethylene. This process produces many other by-products, including propylene, hydrogen, fuel gas, benzene and other organic materials. U.S. Pat. No. 5,763,725 (Choudhary et al.) is an example; reaction temperatures for this conversion range up to 1200° C. and result in significant coking of the reactor, necessitating monthly or bi-monthly cleaning of the reactor. The high temperatures used in conventional steam cracker furnaces also result in excessive production of undesirable nitrous oxides that are a major source of air pollution.

An embodiment of the present invention utilizes a novel catalyst to adiabatically convert ethane to ethylene and other alkenes in a process that operates at much lower temperatures (650° C.-1000° C.) than conventional steam crackers. Operation at reduced temperatures has the advantages of significantly reducing downtime from coking and also reducing the production of nitrous oxides. An embodiment of the present invention allows for reactor designs that are much more compact and have lower construction cost due to materials required for low high temperature operation compared to high temperature operation.

The catalyst families of the present invention, perovskites, are a large family of crystalline ceramics that derive their name from a specific mineral known as perovskite. The parent material, perovskite, was first described in the 1830's by the geologist Gustav Rose, who named it after the famous Russian mineralogist Count Lev Aleksevich von Perovski.

Perovskite-type catalysts include a broad range of compounds in a specific crystalline structure. Perovskite catalysts have been shown to produce synthetic gas (carbon monoxide and hydrogen) from methane. U.S. Pat. No. 5,447,705 (Petit et al.) discloses a catalyst to produce mainly carbon monoxide and hydrogen during the partial oxidation of methane or a gaseous mixture containing methane, such as natural gas or gas combined with oil.

U.S. Pat. No. 5,149,516 (Han et al.) describes partial oxidation of methane over perovskite catalyst wherein methane and oxygen are contacted with the perovskite under conditions sufficient to convert the methane and oxygen to a mixture of carbon monoxide and hydrogen.

U.S. Pat. No. 4,522,706 (Wheelock et al.) describes the use of perovskite containing catalyst in the fluid coking process.

U.S. Pat. Nos. 4,208,269 and 4,179,409 disclose perovskite catalysts and their use in hydrocarbon cracking processes.

U.S. Pat. Nos. 4,055,513 and 4,102,777 disclose high surface area perovskite catalysts and their use in hydrocarbon conversion processes.

The principal perovskite structure found in ferroelectric materials is a simple cubic structure containing three different ions of the form ABO₃. The A and B atoms represent cations having a +2 and +4 valence, respectively, while the O atom is an oxygen having a valence of minus 2 (−2) (See FIG. 1).

Further details of perovskites can be found at Encyclopedia of Crystal Structures (Mat.Sci. 102, Fall, 1999, Univ. Calif., Berkeley). A more complete description of the perovskite structure can be found in Structural Inorganic Chemistry, A. F. Wells, 3rd Edition (Clarendon Press, Oxford, UK, 1962, pages 494 to 499).

Perovskite catalysts have been utilized in oxidative coupling by which process in the presence of an oxidizing agent, the methane is converted at high temperature into higher hydrocarbons, particularly ethane and ethylene, over a suitable catalyst. The oxidizing agent generally used for this purpose is oxygen or air (which generally comprises about 21% oxygen). A novel aspect of the present invention is that the use of steam and/or water introduces additional oxygen and hydrogen to the reaction (as an “enriched air” source) as well as serving to control temperatures. The present invention does not require expensive cryogenic air separation plants to operate efficiently. Use of enriched air sourced from less costly membrane separation units is contemplated. By “enriched air”, applicants are referring to using a feed gas mixture whose oxygen content is greater than that normally found in air, (that is, greater than about 21% oxygen).

Catalysts which exhibit activity in methane oxidative coupling processes are generally formed from metal oxides, and in particular are known catalysts containing oxides of transition metals or metals such as lead, bismuth, tin or antimony, catalysts in the form of strongly basic oxides such as magnesium or calcium oxides doped with alkaline metals, or catalysts containing rare earth elements (see, for example, the descriptions in U.S. Pat. Nos. 4,499,322, 4,499,323, 4,499,324 and 4,495,374, and EP applications 0 177 327 A1 and 0 230 769 A1). The catalysts described in these references do not have the high conversions and selectivity that are produced using embodiments of the present invention.

The literature describes catalysts containing an alkaline metal oxide, an alkaline earth metal oxide, plus possibly one or more transition or rare earth metal oxides that are used in methane oxidative coupling processes (see, for example, Z. K. Bi Yingli et al. Applied Catalysis, 39 (1988) pp 185-190, EP 0 196,541 A1 and U.S. Pat. No. 4,780,449). If the alkaline earth metal is lithium, these catalysts have high initial activity in methane oxidative coupling processes, but this activity falls rapidly over time because of the loss of lithium from the catalyst.

Disadvantages of existing catalytic conversion of paraffinic hydrocarbon, as mentioned previously, include coking of the reactor, production of undesirable nitrous oxides, use of cryogenically produced oxygen, and low yields and conversion. Although significant advances in the field of paraffinic hydrocarbon conversion to alkenes have been made, there still exists a need for better catalysts and processes for converting paraffinic hydrocarbons, particularly for methane and ethane conversion that are capable of providing high conversions and yields over prolonged periods without the use of cryogenic oxygen, without the formation of coke, and with high space velocities and relatively low temperatures and pressures.

### BRIEF DESCRIPTION OF THE SEVERAL VIEWS OF THE DRAWING

FIG. 1 illustrates the basic perovskite crystal structure.

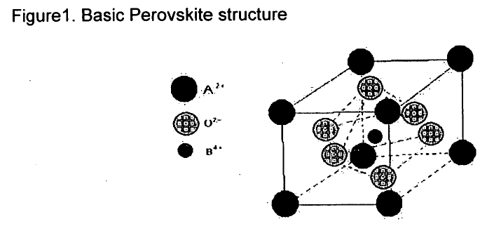

FIG. 2 illustrates the temperature ramp-up process.

FIG. 3 is a schematic diagram of the reactor.

FIG. 4 illustrates the conversion of hydrocarbons to alkenes over a period of time. TOS=time on stream, in minutes.

FIG. 5 is a gas chromatograph tracing of the reactor feed gas mixture (top panel) and of the liquid condensate obtained from the reactor outlet (bottom panel).

FIG. 6 illustrates the conversion of hydrocarbons to alkenes over a period of time with varying ratios of feedstock gasses. TOS=time on stream, in minutes.

### DETAILED DESCRIPTION OF THE INVENTION

Previous processes for converting methane and ethane to higher hydrocarbon compounds have suffered from either low conversion rates and/or low yields; catalyst life has also been limited. The present invention improves on these aspects of low molecular weight paraffinic hydrocarbon (particularly with carbon numbers 1 through 8) conversion to alkenes.

In accordance therewith, for converting methane into hydrocarbon products the present invention provides a perovskite catalyst and process for conversion of paraffinic hydrocarbons into alkenes at high space velocities, low production of coke and long catalyst life without the production of nitrous oxides. The present disclosed family of perovskite catalysts comprising the metals Ti, Sm and Ba (titanium, samarium and barium, respectively) have not previously been recognized as good catalysts for conversion of paraffinic hydrocarbons to alkenes.

The presently disclosed catalysts are highly active for catalyzing the conversion of paraffinic hydrocarbons to alkenes with very high selectivities and yields.

Also provided is a method for making catalysts for the conversion of paraffinic hydrocarbons to alkenes.

Catalysts particularly useful in converting ethane to ethylene and converting methane to ethylene allow for further downstream processing of ethylene into liquids for easier transportation from remote areas.

The catalyst of the present invention is formed by using a sol-gel technique X with specific metal ratios. The metals are dissolved in organic acids to form organo metallic compounds that are gelled.

Suitable organic acids include Formic acid, Acetic acid, Trichloroacetic acid, Dichloroacetic acid, Oxalic acid, Acetoacetic acid, Bromoacetic acid, Chloroacetic acid, lodoacetic acid, Phenylacetic acid, Thioacetic acid, Glycolic acid, Cacodylic acid, Cyanoacetic acid, Acrylic acid, Pyruvic acid, Malonic acid, Propanoic acid, Chloropropanoic acid, Hydroxypropanoic acid, Lactic acid, Glyceric acid, Cysteic acid, Barbituric acid, Alloxanic acid, Maleic acid, Oxaloacetic acid, Methymalonic acid, Malic acid, Tartaric acid, Dihydroxytartaric acid, Butanoic acid, Hydroxybutanoic acid, Chlorobutanoic acid, Aspartic acid, Itaconic acid, Mesaconic acid, Dimethylmalonic acid, Glutaric acid, Succinic acid, Methylsuccinic acid, L-Glutamic acid, Diaminopimelic acid, Pentanoic acid, Trimethylacetic acid, Picric acid, Picolinic acid, Pyridinecarboxylic acid, Benzenesulfonic acid, Aminobenzenesulfonic acid, Ascorbic acid, Citric acid, Isocitric acid, Carboxyglutamic acid, Adipic acid, Adiparnic acid, Hexanoic acid, Benzoic acid, Hydroxybenzoic acid, Dihydroxybenzoic acid, Bromobenzoic acid, Chlorobenzoic acid, lodobenzoic acid, Dinicotinic acid, Dipicolinic acid, Lutidinic acid, Nitrobenzoic acid, Quinolinic acid, Dihydroxymalic acid, Gallic acid, Aminobenzoic acid, Cyclohexanecarboxylic acid, Heptanedioic acid, Ethylglutamic acid, Heptanoic acid, Phthalic acid, Terephthalic acid, Chlorophenylacetic acid, Nitrophenylacetic acid, Toluic acid, Homogentisic acid, Octanoic acid, Chlorocinnamic acid, Cyanophenoxyacetic acid, Cinnamic acid, Hippuric acid, Mesitylenic acid, Nonanic acid, Methylcinnamic acid, Naphthoic acid, Tridecylamine, and Diphenylacetic acid.

The catalyst composition comprises three metals: a Group 2 metal of the periodic table of the elements, most preferably barium; a Group IV transition metal of the periodic table of the elements, most preferably titanium, and a lanthanoids group element. The lanthanoid element is chosen from the group consisting of samarium (Sm), rhodium (Rh) or ruthenium (Ru), due to their high melting points and ability to form perovskite crystals with a tolerance factor close to 1. Other metals such as tin (Sn) can be used but suffer from short catalyst life due to their depletion under the high temperatures experienced under the reaction conditions employed for conversion of paraffinic hydrocarbons to alkenes. The tin component of the catalyst can also be extracted because of the chlorides that are used to extend catalyst activity.

A number of different metal salts can be used to produce the catalyst. Among the barium salts are barium acetate Ba(C₂H₃O₂), barium bromide (BaBr₂.2 H₂O), barium chloride (BaCl₂.2 H₂O), barium nitrite Ba(NO₂)₂, barium nitrate (BaNO₃)₂, barium oxide (BaO) and barium sulfate (BaSO₄). The titanium salts can be chosen from titanium IV chloride (TiCl₄), titanium dioxide (TiO₂) and titanium sulfate (TiSO₄). Other Group IV transition metals, such as zirconium, (Zr) could also be used. The metals should be in an oxide or other salt form in order to react with the organic acid to form organo metallic compounds. Preferably the barium should be in the form of barium chloride (BaCl₂) or barium oxide (BaO), the titanium in the form of titanium IV chloride (TiCl₄) or titanium dioxide (TiO₂) and the samarium in the form of samarium chloride (SmCl₃) or samarium oxide (Sm₂O₃). It has been discovered that the formation of organo metallic compounds followed by specific calcination conditions results in a more active and sustainable catalyst for the conversion of paraffinic hydrocarbons into alkenes.

The general formula of the perovskite catalyst composition is represented as ABX₃ where ‘A’ and ‘B’ are cations and X is an anion. The ‘A’ and ‘B’ atoms represent ions having a valence of +2 and +4, respectively, while the ‘X’ atom is an anion with a valence of minus 2 (−2), such as oxygen.

‘A’ comprises a lanthanoid metal and an alkaline earth metal (Group II metals) in the lattice and ‘B’ is a Group IV transition metal cation. In addition to the ABX structure described above, the catalyst can be formed as Aₓ B_{y}Ti_{z}, where ‘x’ is equal to about 0.2 to 1; ‘y’ is equal to about 1 to 2 and ‘z’ is equal to about 1. In this instance, the formula for this embodiment of the perovskite composition can be represented as Ba(₂₋ₓ)SmₓTiO₃, if x=0.2. If x=1.0, the formula of the perovskite composition can be represented as Ba₍₅ₓ₎Sm_{(1.5x)}TiO₃.

Calcination of the dried organo metallic gel is most preferably performed using a ramped temperature profile where each temperature step is held for ¼ h starting at 200° C., hold, increase the temperature to about 400° C., hold, increase the temperature to about 600° C., hold, and then ramp up to a calcination temperature of about 700° C.-1000° C. preferably 750°-850° C. The calcined powder was pressed and sieved as appropriate for the size reactor being used.

The catalyst can be deposited on conventional supports such as, but not intended to be limited to, SiO₂ (silicon dioxide) or Al₂O₃ (aluminum oxide). Although these supports are not essential they may be used to give the catalyst shape and improved mechanical strength. In addition, basic supports such as MgO, CaO and BaO; acidic supports such as a mixture of Al₂O₃ and SiO₂ or zeolites; neutral supports such as MgAl₂O₄, MgCr₂O₄, ZrCrO₄ and ZnAl₂O₄; and amphoteric supports such as alpha-Al₂O₃, TiO₂, CeO₂, and ZrO₂ could be utilized. If certain conventional catalyst supports are used their acidity should be reduced so that the support will not catalyze the formation of carbon oxides.

The catalyst prepared by the method described above is then utilized in a reactor where surfaces are constructed of non-reactive materials such as quartz. It has been discovered that reactor materials such as stainless steel results in undesirable side reactions under the operating conditions of this process.

Another embodiment of the present invention is the discovery that catalyst activity decreases over time and that the intermittent addition of a chloride compound, such as in the form of either carbon tetrachloride (CCl₄) or chloroform (CHCl₃), has the ability to maintain catalyst activity. Chlorine itself can also be used to extend catalyst activity. Additional sources of chlorine include methane chloride (CH₃Cl), ethane chloride, (C₂H₅Cl), methylene chloride, ethylene chloride, vinyl chloride, stannous chloride (SnCl₂), organic or inorganic chlorides and/or hydrochloric acid (HCl).

It has further been discovered that catalyst activity may be further enhanced by the addition of a neutralizing base, such as an amine, following addition of the chloride compound. This base can be selected from the group consisting of methyl amine, dimethyl amine, trimethyl amine, ethyl amine, diethyl amine, triethyl amine, dimethyl ethyl amine, ammonia, and ammonium salts. Ammonia has been demonstrated to be a neutralizing amine.

Another embodiment of the present invention is the use of air, enriched air or oxygen as a source of oxygen to feed into the reactor with paraffinic hydrocarbons such as ethane in a quantity sufficient to result in formation of the desired end product. When ethane is the reactant the range of molar ratio of oxygen (O₂) to ethane is 1:1 to 1:9 and preferably 1:2 to 1:5 and more preferably 1:2 to 1:4. When methane is the reactant the preferred ratio of oxygen (O₂) to methane is about 1:2. Excess oxygen addition to the feed gas can result in increased production of undesirable carbon monoxide (CO) and carbon dioxide (CO₂).

Yet another embodiment of the present invention is the conversion of ethane to ethylene, conducted under autothermal reaction conditions wherein the feed gas is partially combusted, and the heat produced during combustion drives the endothermic cracking process, thus requiring no external heat source for the reaction.

The temperature of the reactor affects the process of the present invention. Temperature control may involve cooling when the reaction is exothermic, such as when methane is converted to ethylene. Endothermic reactions, such as conversion of ethane to ethylene, require a heat source that can be provided by the oxidation of a portion of the feedstock. In certain embodiments of the present invention, the reactor may or may not utilize cooling coils or steam injection for temperature control, and steam injection as a source of oxygen and hydrogen. The catalyst may also be used in either a fixed bed, or fluid bed design. A large fixed bed reactor with interstage cooling or cold-shot injection may also be used in embodiments of the present invention.

Another embodiment of the present invention is operation of the process under pressure. It is preferably operated without applying higher than atmospheric pressure, at a temperature generally from about 650° C. to about 1000° C., preferably from about 750° C. to about 950° C., and more preferably from about 780° C. to about 850° C.

Yet another embodiment of the present invention is the operation of the reactor either with or without preheating of the feed gases.

It is also envisioned that commercially available technology can be utilized to recycle unconverted reactor outlet product back to the inlet feed of the reactor for further conversion, thus providing yet higher yields for the process. It is also envisioned that the reactor outlet products can be used in commercially available processes that utilize mixed gas streams to produce still higher value products.

While embodiments of the present invention have been shown and described, they are exemplary only, and are not intended to be limiting except as defined in the claims, and with the understanding that modifications of these embodiments can be made by one skilled in the art without departing from the spirit and scope of the present invention. The disclosures of patents, patent applications and publications cited herein are hereby incorporated by reference. The discussion of certain patents, patent applications and publications is not to be construed that they are prior art.

The following experimental examples are provided.

### EXAMPLE 1

Sol-gel Method of Catalyst Production.

A perovskite catalyst was prepared using the sol-gel technique. The following reagent grade materials were used: TiCl₄ (titanium chloride), BaO (barium oxide) and Sm₂O₃ (samarium oxide) (all from Aldrich, Milwaukee, Wis.). Propanoic acid (Across Chemical, division of Ranbaxy Laboratories, India) was used as the organic acid. The ‘t’ factor for this catalyst formulation was calculated to be within the desired range (approximately=1)

Twenty-five (25) grams of TiCl₄; 14 grams Sm₂O₃ and 28 grams of BaO were placed in separate glass flasks with enough organic acid (between 400 and 1000 ml) to dissolve the salts. Each flask was equipped with reflux condensers. The solutions were heated with an electric mantle until boiling. The mixtures were boiled until the oxides and salts were dissolved, thus forming individual organo-metallic solutions (approximately 2-5 hours at a temperature between 90° C.-140 ° C.).

The solutions were then combined in a ratio such that the moles of metal content equated to 1.5 moles barium; 1 mole titanium; and 0.5 moles samarium.

The ratios of the metals employed in the formation of the perovskite catalyst can include barium in ratios ranging from about 1 mole to about 2 moles, and samarium ranging from about 0.1 to about 1 mole, with titanium generally being used at about 1 mole.

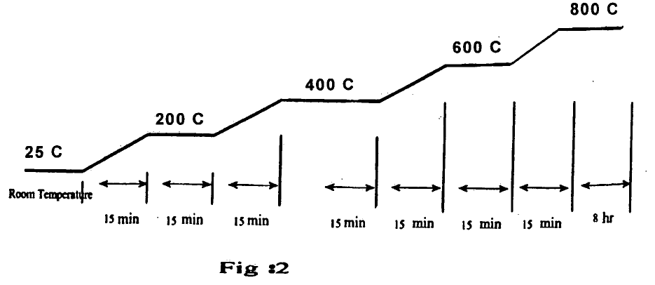

The resulting mixture of solutions was then heated without reflux to evaporate the excess liquid until a thick gel formed (approximately 2-3 hours). The gel was dried, crushed, and the powder placed in a ceramic tray in an electrically heated furnace where it was calcined according to the temperature profile outlined in FIG. 2 to produce a perovskite catalyst.

As shown in FIG. 2 (not to scale), temperature ramp increases of approximately 200° C. occur over about ¼-½ hour (h) followed by a holding period of a similar ¼-½ hour until a target temperature in the range from about 700° C.-about 1000° C. is reached, preferably about 800° C. The powdered material is subjected to the final calcination temperature for an additional period of about 8 hours or more during which time calcination occurs. Accordingly, in one embodiment of the present invention, starting from a room temperature (ambient temperature) of about 25° C., 7 steps of ¼ hour each will result in a final calcination temperature of about 800 ° C. in about 1 and ¾ hours.

After calcining, the calcined material is pressed and pelletized under 5.5 tons/cm² and crushed into 1.98 mm ˜3.96 mm particles followed by sieving to select a powder having a size compatible for use with a fixed-bed reactor used for the partial oxidation of methane to produce ethylene.

The following measurement techniques and definitions apply to the examples that follow:
1. Liquid flow rates were metered by use of syringe pumps and/or positive displacement pumps. In each case the pumps were calibrated for the particular flow settings.
2. Gas flow rates were measured with mass flow meters and reported as gas flow rates at 0° C. and 1 atmosphere (101.325 kPa).
3. Composition of the gas feed was calculated based on flow rates determined from the mass flow meters. The gas composition as measured by gas chromatography was also determined to be the same as the composition calculated from the mass flow meters.
4. The composition of the exit gases from the reactor was measured by gas chromatography using a gas chromatograph calibrated with standard gas mixtures. An internal standard of nitrogen was used to calculate the exit flow rate of the gas from the reactor.
5. Condensed water from the reactor was collected and measured gravimetrically. The calculated wet basis measurements were based on including the water reactant/products in the total reactant weight. Dry basis measurements were calculated by eliminating all water from the reactor outlet and then calculating molar percentage.
6. Temperatures in the reactor were measured by use of a thermocouple that could be moved up and down within a thermowell inserted into the center of the reactor.
7. Space velocities were calculated as volumetric feed rate of the total feed calculated at 0° C. and 1 atmosphere (101.325 kPa) calculated as a gas flow rate at 0° C. and one atmosphere (101.325 kPa), divided by the volume of the catalyst. In some cases weight hourly space velocities (“WHSV”) are reported and these were based either on the total mass flow rate of the feed divided by the mass of catalyst or the mass flow rate of methane divided by the total mass of catalyst.
8. Conversion (“Conv”) of methane (or ethane) is calculated as the moles of methane fed minus the moles of methane (or ethane) in the reactor exit and this difference is divided by the methane (or ethane) fed. The percent conversion is 100 times the fractional conversion.
9. Selectivity (“Sel”) is calculated by two methods: 1) Utilizing the total flow rate that is calculated using nitrogen as a tie component, i.e., forcing a nitrogen balance and the exit composition of the gas leaving the reactor. The ethylene produced times 2 divided by the methane consumed is equal to the ethylene selectivity. 2) The second method forces a carbon balance and calculates the selectivity from only the exit composition of the gas from the reactor, thereby forcing a carbon balance. These two methods should give the same results unless there are measurement or analytical errors (or carbon deposits in the reactor or on the catalyst. The two methods gave an indication of the error in the measurements and the assumption that there is no coking within the reactor. In the case of hydrocarbon feeds having carbon numbers greater than that of propane, the differences in selectivities indicate accumulation of carbon, in the form of coke, inside the reactor.
10. Yield for a single pass reactor system is the product of the conversion times the selectivity. Yield=(conversion)×(selectivity)=moles of component ‘I’ produced times the number of carbons in the component divided by the moles of methane fed. Ultimate yield for a process with recycle is equal to the selectivity for the single pass reactor experiments because for a recycle process, un-reacted methane Is recycled to extinction such that the conversion of the reactant molecule to the process is 100%.

Yield=(Conv)(Select)

### EXAMPLE 2

Use of Catalyst to Convert Hydrocarbons to Alkenes.

The catalyst of Example 1 was used to illustrate the effectiveness of a catalyst produced by the sol-gel technique.

The following equipment and materials were used in this example:

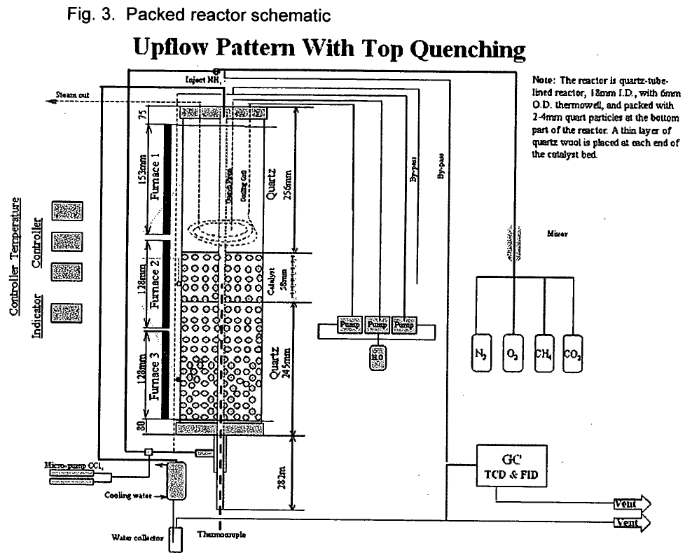

Reactor: The reactor is a quartz-lined, SS304L tube with an inside diameter of 18 mm with a 6 mm outside diameter quartz thermowell at the reactor center. About 10 grams of catalyst was charged to the reactor. The reactor configuration is shown in FIG. 3, in which the catalyst employed is packed between a layer of quartz on the top and bottom of the catalyst bed. Also shown are three separate furnaces used to heat the reactor, with two of the furnaces heating the catalyst bed.

The reactor was heated with three independent furnaces at the top, middle and bottom sections. The reactor was heated up to 450° C. with nitrogen flow at about 100 ml/min. At 450° C. and above, the reactor was heated up with the reactant mixture, the composition of which is indicated in Table 1; data from duplicate experiments (“Expt”) are shown. Volumetric flowrates that are given in Table 1 are for 0° C. and 1 atmosphere.

TABLE 1
Reactor inlet feeds

| Gas Feeds | Expt. A | Expt. B |
|---|---|---|
| N₂, ml/min | 301.1 | 301.1 |
| O₂, ml/min | 151.2 | 151.2 |
| CH₄, ml/min | 242.7 | 242.7 |
| C₂H₆, ml/min | 60.6 | 60.6 |
| Steam, g/hr. | 58.3 | 58.3 |

TABLE 2
Results of Conversion of Hydrocarbons to Alkenes

| | CH₄ | C₂H₄ | C₂H₆ | COx | C₂H₄ | CO | C2+ | C2+ |
|---|---|---|---|---|---|---|---|---|
| Expt. | conversion | selectivity | conversion | selectivity | yield | selectivity | yield | selectivity |
| No. | (%) | (%) | (%) | (%) | (%) | (%) | (%) | (%) |
| A | 29.30 | 49.28 | 61.49 | 32.36 | 19.72 | 4.59 | 21.94 | 54.83 |
| B | 31.34 | 48.78 | 66.80 | 34.28 | 21.05 | 4.09 | 23.68 | 54.89 |

The reaction conditions were:
Gas Hourly Space Velocity (GHSV) for both experiments A and B was 13,104.
Catalyst weight was ˜10 gms.
Maximum reactor temperature was 867° C.
Carbon tetrachloride (CCl₄) was injected at a rate of 40 microliters (μl)/hr.
Selectivity and yields are on a carbon basis.

The results in Table 2 show that the catalyst and process are effective in converting methane and ethane into alkenes. Although the example indicates yields for single pass conversions it is anticipated that recycling of unreacted feed gas compounds will result in even higher overall conversion and yields.

### EXAMPLE 3

Effect of Reaction Conditions on Conversion of Hydrocarbons to Alkenes.

Table 3 summarizes a series of experiments, using the reactor configuration described in Example 2. For this series of experiments the GHSV ranged between 1175 and 7037.

This series of tests was conducted over a range of operating conditions. The data revealed that reaction temperature affects methane conversion and C₂ selectivity. If the temperature is too high, such as when it exceeds 900° C., conversion activity decreases due to deactivation of the catalyst. If the temperature is too low, no reaction will occur. The reaction temperature range is from about 750° C.˜825° C. in the catalyst bed. The hotspot temperature in the catalyst bed should be below about 835° C. to protect the catalyst and to maintain the C₂+ yield. Lower reaction temperatures give a higher C₂ selectivity but a lower methane conversion; higher reaction temperatures give a higher methane conversion but lower C₂ selectivity. The initial temperature to convert methane is about 650° C.

TABLE 3
Conversion Yield and Selectivity Data

| | | | | | | | | | | Feed Gas |
|---|---|---|---|---|---|---|---|---|---|---|
| | | O₂ | | | | C₂ | C₂H₄ | C2+ | | (N₂; O₂; |
| Expt. | CH₄ | conv | C₂ | C₂H₄ | COx | yield | yield | yield | C2+ | CH₄; CO₂; H₂O |
| # | conv % | % | sel, % | sel % | sel % | % | % | % | sel | ml/min) |
| 1 | 47 | 97 | 48 | 38 | 40 | 23 | 18 | 25 | 53 | 112; 117; 117; 117; 11 |
| 2 | 46 | 97 | 50 | 38 | 41 | 23 | 17 | 25 | 54 | 20.; 101; 203; 0; 19 |
| 3 | 37 | 78 | 60 | 53 | 27 | 22 | 19 | 24 | 66 | 58; 55; 109; 391; 0 |
| 4 | 44 | 100 | 48 | 43 | 49 | 21 | 19 | 24 | 56 | 57; 55; 112; 112; 0 |
| 5 | 35 | 79 | 63 | 56 | 31 | 22 | 20 | 24 | 69 | 58; 55; 109; 391; 0 |
| 6 | 40 | 91 | 55 | 47 | 38 | 22 | 19 | 24 | 61 | 120; 117; 240; 236; 0 |
| 7 | 37 | 83 | 60 | 53 | 36 | 22 | 19 | 24 | 66 | 388; 137; 275; 685; 0 |
| 8 | 45 | 91 | 50 | 36 | 39 | 22 | 16 | 24 | 54 | 203; 101; 203; 0; 39 |
| 9 | 43 | 79 | 52 | 42 | 40 | 22 | 18 | 24 | 56 | 730; 212; 424; 0; 0 |
| 10 | 39 | 82 | 58 | 47 | 53 | 23 | 19 | 24 | 61 | 425; 212; 424; 0; 0 |
| 11 | 40 | 88 | 55 | 44 | 53 | 22 | 18 | 24 | 60 | 425; 212; 424; 0; 0 |
| 12 | 39 | 85 | 56 | 45 | 53 | 22 | 18 | 24 | 61 | 425; 212; 424; 0; 0 |
| 13 | 44 | 99 | 47 | 42 | 49 | 21 | 19 | 24 | 55 | 425; 212; 424; 0; 0 |

### EXAMPLE 4

Effectiveness for Conversion of a Feed Gas Mixture of Ethane and Methane.

To test the effectiveness of the catalyst and process in converting a feed gas mixture of ethane and methane, the reactor configuration from Example 2 was used. The catalyst was prepared using the procedure described in Example 1. The maximum reactor temperature was about 845° C.

Table 4 summarizes the feed gas to the reactor, and the reactor outlet composition is shown in Table 5. In Table 5 the three columns dealing with mole per cent are as follows: first column is the reactor outlet on a wet basis; second is the reactor outlet on a dry basis. The determination of wet and dry basis has been described in Example 1. The third column refers only to products produced on a dry basis, i.e. Columns 1 and 2 for example contain ethane, but column three does not.

TABLE 4
Inlet Gas Feed

| Feed Gas | |
|---|---|
| N₂, ml/min | 259.4 |
| O₂, ml/min | 129.3 |
| CH₄, ml/min | 225.3 |
| C₂H₆, ml/min | 39.1 |
| Steam, g/hr | 12.5 |

TABLE 5
Reactor outlet analysis

| | | Reactor Outlet | | |
|---|---|---|---|---|
| | rate, | mol % | mol % | Product (dry-base) |
| Component | mol/hr | (wet) | (dry) | mol % |
| H₂ | 0.0419 | 1.77 | 2.94 | 17.35 |
| O₂ | 0.0511 | 2.16 | 3.58 | 0.00 |
| N₂ | 0.6945 | 29.34 | 48.65 | 0.00 |
| CO | 0.0253 | 1.07 | 1.77 | 10.46 |
| CH₄ | 0.4112 | 17.37 | 28.80 | 0.00 |
| CO₂ | 0.0965 | 4.08 | 6.76 | 39.95 |
| C₂H₄ | 0.0725 | 3.06 | 5.08 | 30.00 |
| C₂H₆ | 0.0291 | 1.23 | 2.04 | 0.00 |
| C₃H₈ | 0.0008 | 0.03 | 0.05 | 0.32 |
| C₃H₆ | 0.0026 | 0.11 | 0.18 | 1.06 |
| I—C₄H₁₀ | 0.0003 | 0.01 | 0.02 | 0.12 |
| N—C₄H₁₀ | 0.0000 | 0.00 | 0.00 | 0.01 |
| C₄H₈ | 0.0014 | 0.06 | 0.10 | 0.60 |
| C₅H₁₂ | 0.0001 | 0.01 | 0.01 | 0.06 |
| C₅H₁₀ | 0.0000 | 0.00 | 0.00 | 0.00 |
| C₆+Nonaromatic | 0.0000 | 0.00 | 0.00 | 0.02 |
| Benzene | 0.0001 | 0.00 | 0.01 | 0.05 |
| Toluene | 0.0000 | 0.00 | 0.00 | 0.01 |
| Xylene | 0.0000 | 0.00 | 0.00 | 0.00 |
| AromC₉₊ | 0.0000 | 0.00 | 0.00 | 0.00 |
| Water | 0.9396 | 39.69 | | |

The reaction conditions were:
The reactor flow rate was GHSV=6100.
The weight of catalyst used was 10.20 g.
Amine, in the form of NH₃ was injected at a rate of 6 ml/h.

CCl₄ was injected at a rate of 40 μl/hr.

TABLE 6
Calculated yields and selectivity

| | |
|---|---|
| C₂H₄ Selectivity (%) | 50.75 |
| C₂₊ Selectivity (%) | 57.37 |
| C₂H₄ Yield (%) | 19.20 |
| C₂₊ Yield (%) | 21.70 |

The results show that the catalyst and process are effective in converting a combined feed of methane and ethane into alkenes with high selectivity to ethylene.

### EXAMPLE 5

The catalyst of Example 1 was used with the reactor configuration of Example 2, using a mixture of gas feeds as follows:

TABLE 7
Reactor inlet streams

| Expt | A | B | C |
|---|---|---|---|
| N₂, ml/min | 122.5 | 122.5 | 122.5 |
| O₂, ml/min | 242.6 | 242.6 | 242.6 |
| CH₄, ml/min | 48.4 | 48.4 | 48.4 |
| C₂H₆, ml/min | 437.4 | 437.4 | 437.4 |
| Steam, g/hr | 0.0 | 0.0 | 0.0 |

The reaction conditions were:
GHSV=5500.
Weight of catalyst: 12.03 g.
CCl₄ was injected at 60 μl/hr.
NH₃ was injected at 3 ml/30 min.

The reactor temperature was controlled by a direct steam quench into it to control reactor temperature to a maximum of 845° C.

TABLE 8
Reactor Outlet Analysis Experiment

| | A | | B | | C | |
|---|---|---|---|---|---|---|
| Reactor Oulet | mol/hr. | mol % | mol/hr. | mol % | mol/hr | mol % |
| H₂ | 0.43969 | 15.982 | 0.38713 | 12.790 | 0.37742 | 12.399 |
| O₂ | 0.01961 | 0.713 | 0.05259 | 1.738 | 0.03553 | 1.167 |
| N₂ | 0.32798 | 11.921 | 0.32798 | 10.836 | 0.32798 | 10.774 |
| CO | 0.21129 | 7.680 | 0.18271 | 6.036 | 0.18553 | 6.095 |
| CH₄ | 0.34731 | 12.624 | 0.31201 | 10.308 | 0.31388 | 10.311 |
| CO₂ | 0.17768 | 6.458 | 0.17185 | 5.678 | 0.18310 | 6.015 |
| C₂H₄ | 0.58660 | 21.322 | 0.53940 | 17.821 | 0.55091 | 18.098 |
| C₂H₆ | 0.31664 | 11.509 | 0.38194 | 12.619 | 0.38970 | 12.802 |
| C₃H₈ | 0.00271 | 0.098 | 0.00282 | 0.093 | 0.00281 | 0.092 |
| C₃H₆ | 0.01059 | 0.385 | 0.00964 | 0.319 | 0.01000 | 0.328 |
| I—C₄H₁₀ | 0.00310 | 0.113 | 0.00214 | 0.071 | 0.00165 | 0.054 |
| N—C₄H₁₀ | 0.00016 | 0.006 | 0.00012 | 0.004 | 0.00011 | 0.004 |
| C₄H₈ | 0.00977 | 0.355 | 0.00813 | 0.269 | 0.00827 | 0.272 |
| C₅ | 0.00082 | 0.030 | 0.00068 | 0.023 | 0.00058 | 0.019 |
| C₆+ₙₒₙₐᵣₒₘ | 0.00043 | 0.016 | 0.00032 | 0.010 | 0.00044 | 0.015 |
| Benzene | 0.00178 | 0.065 | 0.00125 | 0.041 | 0.00143 | 0.047 |
| C₇₊ₐᵣₒₘ | 0.00012 | 0.004 | 0.00006 | 0.002 | 0.00010 | 0.003 |
| Water | 0.29493 | 10.720 | 0.64600 | 21.343 | 0.65461 | 21.505 |

TABLE 9
Calculated conversions selectivity and yields.

| Experiment | A | B | C |
|---|---|---|---|
| C₂H₆ conv, % | 72.96 | 67.38 | 66.72 |
| Sel to CO % | 14.17 | 13.09 | 13.46 |
| C₂₊ yield, % | 51.92 | 47.38 | 48.36 |
| C₂H₄ yield, % | 47.46 | 43.65 | 44.58 |

This example shows high selectivity and yield of a mixed (ethane and methane) feed stream to ethylene with additional production of higher carbon (C₂+) alkanes and alkenes when using a mixed gas feed of methane and ethane. Although the example indicates yields for single pass conversions it is anticipated that recycling of unreacted feed gas compounds will result in even higher overall conversion and yields.

### EXAMPLE 6

Effectiveness for Conversion of an Ethane Feed Gas.

To test the effectiveness of the catalyst of the present invention on an ethane feed gas, the catalyst of Example 1 was used in the reactor configuration of Example 2. The reactor feed gas streams were as follows:

TABLE 10
Reactor Feed Gasses

| | |
|---|---|
| N₂, ml/min | 203.8 |
| O₂, ml/min | 101.4 |
| CH₄, ml/min | 0.0 |
| C₂H₆, ml/min | 203.4 |
| Steam, g/hr | 0.0 |

The reaction conditions were:
GHSV=3392 (max).
Weight of catalyst: 9.0 g.
CCl₄ injected at the rate of 40 μl/h.

The reactor outlet gas analyses are shown in Table 11.

TABLE 11
Reactor Outlet analysis

| | |
|---|---|
| Reactor Outlet | Product (dry- |

| | rate, | mol % | mol % | base) |
|---|---|---|---|---|
| Components | mol/hr | (wet) | (dry) | mol % |
| H₂ | 0.2769 | 15.49 | 18.34 | 32.97 |
| O₂ | 0.0000 | 0.00 | 0.00 | 0.00 |
| N₂ | 0.5458 | 30.52 | 36.14 | 0.00 |
| CO | 0.1766 | 9.88 | 11.70 | 21.03 |
| CH₄ | 0.0735 | 4.11 | 4.87 | 8.75 |
| CO₂ | 0.0259 | 1.45 | 1.72 | 3.09 |
| C₂H₄ | 0.2725 | 15.24 | 18.05 | 32.45 |
| C₂H₆ | 0.1244 | 6.96 | 8.24 | 0.00 |
| C₃H₈ | 0.0013 | 0.07 | 0.09 | 0.16 |
| C₃H₆ | 0.0065 | 0.36 | 0.43 | 0.78 |
| I—C₄H₁₀ | 0.0006 | 0.03 | 0.04 | 0.07 |
| N—C₄H₁₀ | 0.0000 | 0.00 | 0.00 | 0.00 |
| C₄H₈ | 0.0054 | 0.30 | 0.35 | 0.64 |
| C₅H₁₂ | 0.0001 | 0.00 | 0.01 | 0.01 |
| C₅H₁₀ | 0.0000 | 0.00 | 0.00 | 0.00 |
| C₆+Nonarom | 0.0001 | 0.00 | 0.00 | 0.01 |
| Benzene | 0.0004 | 0.02 | 0.02 | 0.04 |
| Toluene | 0.0000 | 0.00 | 0.00 | 0.00 |
| Xylene | 0.0000 | 0.00 | 0.00 | 0.00 |
| AromC₉₊ | 0.0000 | 0.00 | 0.00 | 0.00 |
| Water | 0.2780 | 15.55 | | |

TABLE 12
Calculated Selectivity and Yields

| | |
|---|---|
| C₂H₄ Sel, % | 62.51 |
| C₂₊ Sel, % | 68.34 |
| C₂H₄ Yield, % | 48.64 |
| C₂₊ Yield, % | 53.17 |

The data reveals that the catalyst and process are effective in converting ethane to ethylene and higher value alkenes. In this example undesirable carbon dioxide levels are kept low, and a valuable co-product, hydrogen (H₂) gas and carbon monoxide (CO), are also produced.

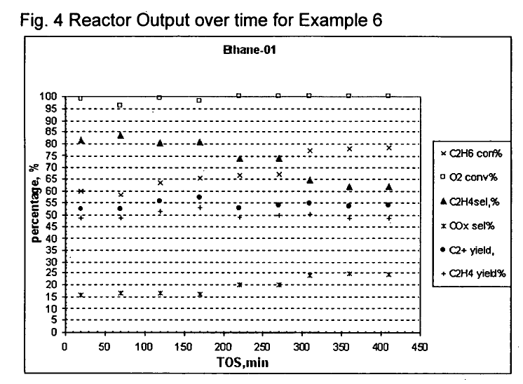

This was run over several hours to test the effectiveness of the process over time. The results are shown in FIG. 4.

### EXAMPLE 7

Effectiveness for conversion of a naphtha-containing hydrocarbon feedstock.

A feedstock of highly paraffinic liquid naphtha derived from a commercial gas to liquids plant (Conoco Phillips Co., Ponca City, Okla.) was used. The reactor inlet feeds are shown in Table 13

TABLE 13
Reactor Inlet stream

| | |
|---|---|
| N₂, ml/min | 203.8 |
| O₂, ml/min | 145.2 |
| CH₄, ml/min | 0.0 |
| Naphtha, ml/hr | 87.8 |
| Steam, g/hr | 0.0 |

The reactor outlet products are summarized in Table 14.

TABLE 14
Reactor outlet analysis

| | Reactor Outlet | | | |
|---|---|---|---|---|
| | | | | Product, |
| | rate, | mol % | mol % | dry base |
| Components | mol/hr | (wet) | (dry) | (mol %) |
| H₂ | 0.4219 | 12.19 | 13.26 | 16.00 |
| O₂ | 0.0000 | 0.00 | 0.00 | 0.00 |
| N₂ | 0.5458 | 15.77 | 17.15 | 0.00 |
| CO | 0.3041 | 8.79 | 9.56 | 11.54 |
| CH₄ | 0.5723 | 16.53 | 17.99 | 21.71 |
| CO₂ | 0.0898 | 2.59 | 2.82 | 3.41 |
| C₂H₄ | 0.7775 | 22.46 | 24.43 | 29.49 |
| C₂H₆ | 0.1036 | 2.99 | 3.26 | 3.93 |
| C₃H₈ | 0.0054 | 0.16 | 0.17 | 0.21 |
| C₃H₆ | 0.1909 | 5.52 | 6.00 | 7.24 |
| I—C₄H₁₀ | 0.0055 | 0.16 | 0.17 | 0.21 |
| N—C₄H₁₀ | 0.0019 | 0.06 | 0.06 | 0.07 |
| C₄H₈ | 0.0825 | 2.38 | 2.59 | 3.13 |
| C₅H₁₂ | 0.0032 | 0.09 | 0.10 | 0.12 |
| C₅H₁₀ | 0.0000 | 0.00 | 0.00 | 0.00 |
| C₆ + Nonarom | 0.0166 | 0.48 | 0.52 | 0.63 |
| Benzene | 0.0518 | 1.50 | 1.63 | 1.96 |
| Toluene | 0.0052 | 0.15 | 0.16 | 0.20 |
| Xylene | 0.0000 | 0.00 | 0.00 | 0.00 |
| C₇₊ Nonarom | 0.0039 | 0.11 | 0.12 | 0.15 |
| Water | 0.2795 | 8.07 | | |

TABLE 15
Calculated Yields

| | |
|---|---|
| Naptha conv | 100.00 |
| Oxy Conv | 100.00 |
| Mass yield of C₂H₄, g/100 g-feed | 33.45 |
| Mass yield of C₃H₆, g/100 g-feed | 12.32 |
| Mass yield of C₄H₈, g/100 g-feed | 7.10 |
| Mass yield of COₓ, g/100 g-feed | 19.15 |

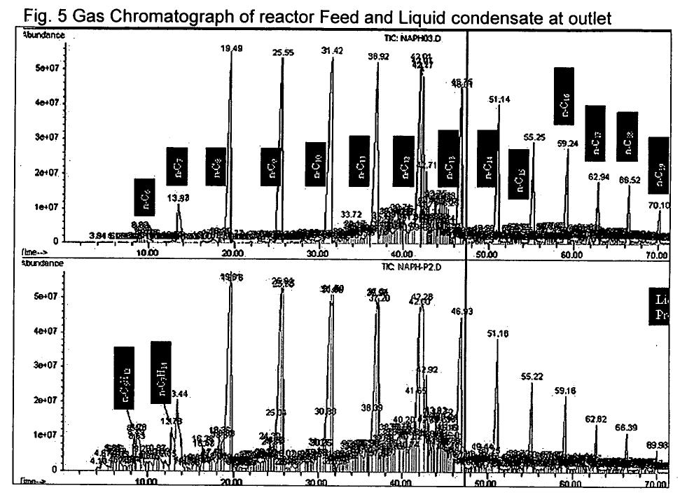

The data indicates the catalyst and process are effective in converting higher carbon alkanes into alkenes. As shown in FIG. 5, the gas chromatographic data shows that the liquid products are primarily unreacted paraffins which can be recycled to the reactor to enhance alkene yield. Any generation of valuable co-products such as hydrogen and carbon monoxide that can be utilized in a variety of downstream processes such as synthetic gas reformation. The data indicates yields for single pass conversions; it is anticipated that recycling of feed gas compounds will result in even higher overall conversion and yields.

### EXAMPLE 8

Effect of an Enriched Air Stream on Conversion of an Ethane Feed Gas.

This example replicated an enriched air stream (that is, enriched with nitrogen and oxygen) combined with ethane (Table16).

TABLE 16
Feed gas to reactor

| | |
|---|---|
| N₂, ml/min | 338.8 |
| O₂, ml/min | 169.1 |
| CH₄, ml/min | 0.0 |
| C₂H₆, ml/min | 423.5 |
| Steam, g/hr | 0.0 |

The output of the reactor was analyzed as described in Example 1, and the data are shown in Table 17.

TABLE 17
Reactor outlet analysis

| | Reactor Outlet | | | |
|---|---|---|---|---|
| | | | | Product |
| | rate, | mol % | mol % | (dry-base) |
| Components | mol/hr | (wet) | (dry) | mol % |
| H₂ | 0.3164 | 10.53 | 12.77 | 29.15 |
| O₂ | 0.0055 | 0.18 | 0.22 | 0.00 |
| N₂ | 0.9070 | 30.19 | 36.60 | 0.00 |
| CO | 0.0966 | 3.22 | 3.90 | 8.90 |
| CH₄ | 0.0957 | 3.18 | 3.86 | 8.81 |
| CO₂ | 0.0990 | 3.30 | 4.00 | 9.12 |
| C₂H₄ | 0.5519 | 18.37 | 22.27 | 50.84 |
| C₂H₆ | 0.3845 | 12.80 | 15.52 | 0.00 |
| C₃H₈ | 0.0025 | 0.08 | 0.10 | 0.23 |
| C₃H₆ | 0.0075 | 0.25 | 0.30 | 0.69 |
| I—C₄H₁₀ | 0.0015 | 0.05 | 0.06 | 0.14 |
| N—C₄H₁₀ | 0.0001 | 0.00 | 0.00 | 0.01 |
| C₄H₈ | 0.0084 | 0.28 | 0.34 | 0.78 |
| C₅H₁₂ | 0.0004 | 0.01 | 0.02 | 0.04 |
| C₅H₁₀ | 0.0000 | 0.00 | 0.00 | 0.00 |
| C₆ + nonarom | 0.0002 | 0.01 | 0.01 | 0.02 |
| Benzene | 0.0010 | 0.03 | 0.04 | 0.09 |
| Toluene | 0.0001 | 0.00 | 0.00 | 0.01 |
| Xylene | 0.0000 | 0.00 | 0.00 | 0.00 |
| AromC₉₊ | 0.0000 | 0.00 | 0.00 | 0.00 |
| Water | 0.5261 | 17.51 | | |

TABLE 18
Calculated yields and selectivities.

| | |
|---|---|
| C₂H₄ Sel, % | 74.86 |
| C₂₊ Sel, % | 80.24 |
| C₂H₄ Yield, % | 49.20 |
| C₂₊ Yield, % | 52.74 |

This Example illustrates the catalyst and process capabilities in converting ethane to ethylene. It also illustrates the capability to form valuable co-products such as hydrogen and carbon monoxide with relatively low concentrations of undesirable carbon dioxide. Although the example measured reactor outlet gases for single pass conversions, it is anticipated that recycling of unreacted feed gas compounds will result in even higher overall conversions and yields.

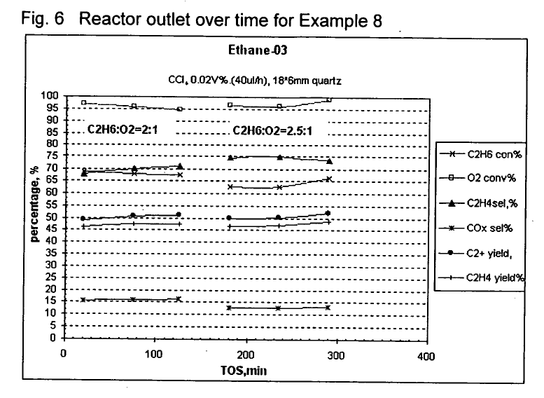

The results in FIG. 6 show the catalyst is active for prolonged periods without deactivation. During the run the ratio of ethane to oxygen was changed as noted in the FIGURE.

What is claimed is:

1. A method of forming a perovskite composition having the general structure of ABX₃, the method comprising the steps of:
preparing a solution comprising:
an alkaline earth metal dissolved in an organic acid, wherein the alkaline earth metal is selected from the group consisting of barium (Ba), magnesium (Mg), calcium (Ca) and strontium (Sr);
a metal selected from Group IV transition metals of the periodic table of the elements dissolved in the organic acid; and
a lanthanoid metal dissolved in the organic acid; wherein the lanthanoid metal is selected from the group consisting of samarium (Sm), rhodium (Rh) and ruthenium (Rh);
heating the solution for a time period sufficient to remove excess organic acid and form a gel;
drying the gel to form a powder;
heating the powder at increasing temperatures in a specified temperature profile; and
calcining the heated powder to form the perovskite composition;
wherein in the composition ‘A’ is the alkaline earth metal and the lanthanoid metal, ‘B’ is the Group IV metal, and X is an anion, and the anion is characterized by having a valence of minus 2 (−2); the alkaline earth metal comprises from about 1 mole to about 2 moles, the Group IV metal comprises about 1 mole, the lanthanoid comprises from about 0.1 mole to about 1.0 moles, and the anion is oxygen.

2. The method as described in claim 1, wherein the organic acid is selected from the group consisting of Propanoic acid, Chloropropanoic acid, Hydroxypropanoic acid, Formic acid, Acetic acid, Trichloroacetic acid, Dichloroacetic acid, Oxalic acid, Acetoacetic acid, Bromoacetic acid, Chloroacetic acid, Iodoacetic acid, Phenylacetic acid, Thioacetic acid, Glycolic acid, Cacodylic acid, Cyanoacetic acid, Acrylic acid, Pyruvic acid, Malonic acid, Lactic acid, Glyceric acid, Cysteic acid, Barbituric acid, Alloxanic acid, Maleic acid, Oxaloacetic acid, Methymalonic acid, Malic acid, Tartaric acid, Dihydroxytartaric acid, Butanoic acid, Hydroxybutanoic acid, Chlorobutanoic acid, Aspartic acid, Itaconic acid, Mesaconic acid, Dimethylmalonic acid, Glutaric acid, Succinic acid, Methylsuccinic acid, L-Glutamic acid, Diaminopimelic acid, Pentanoic acid, Trimethylacetic acid, Picric acid, Picolinic acid, Pyridinecarboxylic acid, Benzenesulfonic acid, Aminobenzenesulfonic acid, Ascorbic acid, Citric acid, Isocitric acid, Carboxyglutamic acid, Adipic acid, Adiparnic acid, Hexanoic acid, Benzoic acid, Hydroxybenzoic acid, Dihydroxybenzoic acid, Bromobenzoic acid, Chlorobenzoic acid, Iodobenzoic acid, Dinicotinic acid, Dipicolinic acid, Lutidinic acid, Nitrobenzoic acid, Quinolinic acid, Dihydroxymalic acid, Gallic acid, Aminobenzoic acid, Cyclohexanecarboxylic acid, Heptanedioic acid, Ethylglutamic acid, Heptanoic acid, Phthalic acid, Terephthalic acid, Chlorophenylacetic acid, Nitrophenylacetic acid, Toluic acid, Homogentisic acid, Octanoic acid, Chlorocinnamic acid, Cyanophenoxyacetic acid, Cinnamic acid, Hippuric acid, Mesitylenic acid, Nonanic acid, Methylcinnamic acid, Naphthoic acid, Tridecylamine, and Diphenylacetic acid.

3. The method as described in claim 1, wherein the organic acid is selected from the group consisting of Propanoic Acid, Formic Acid, Chloropropanoic acid and Hydroxypropanoic acid.

4. The method as described in claim 1, wherein the group IV metal is titanium.

5. The method as described in claim 1, wherein the lanthanoid metal is samarium.

6. The method as described in claim 1, wherein the composition is characterized by having a tolerance factor (“t”) ranging from about 0.8 to about 1.1, wherein t is defined by the equation:
t=(rₐ+rₒ)/(√2 (r_{b}+rₒ))
wherein in the crystal structure of the composition, rₐ and r_{b} are the ionic radii of cation species A and B, respectively, and rₒ is the ionic radius of the anion species.

7 The method as described in claim 5, wherein the samarium is chosen from the group consisting of samarium oxide (Sm₂O₃) and samarium chloride (SmCl₃).

8. The method as described in claim 1, wherein the powder is heated by raising the temperature of the powder in a temperature profile comprising a series of successive ramping and holding stages up to the calcining temperature, and wherein the temperature is raised to the calcining temperature at an increasing rate from about 200° C. to about 400° C. per hour.

9. The method as described in claim 8, wherein the temperature profile further comprises:
ramping the temperature from ambient to about 200° C. for about ¼ hour and holding for a hold period;
ramping the temperature to about 400° C. over about a further ¼ hour and holding for a hold period;
ramping the temperature to about 600° C. over about a further ¼ hour and holding for a hold period; and
ramping the temperature to the calcination temperature over about a further ¼ hour and holding about 8 hours to about 24 hours.

10. The method as described in claim 9, wherein the hold period comprises about ¼ hour to ½ hour.

11. The method as described in claim 9, wherein the calcining temperature ranges from about 700° C. to about 1000° C.

12. The method as described in claim 10, further comprising the step of adding a support agent to the composition, and wherein the support agent is selected from the group consisting of silicon dioxide (SiO₂), aluminum oxide (Al₂O₃), a mixture or silicon dioxide (SiO₂) and aluminum oxide (Al₂O₃), zeolites, magnesium oxide (MgO), barium oxide (BaO), calcium oxide (CaO), magnesium aluminum oxide (MgAl₂O₄), magnesium chromium dioxide (MgCr₂O₄), zirconium chromate (ZrCrO₄), zirconium aluminum oxide (ZrAl₂O₄), alpha-aluminum oxide (α-Al₂O₃), titanium oxide (TiO₂), cerium oxide (CeO₂) and zirconium oxide (ZrO₂).

13. A perovskite composition having the general structure of ABX₃, wherein
A is an alkaline earth metal and a lanthanoid metal, wherein the alkaline earth metal is chosen from the group consisting of barium (Ba), magnesium (Mg), calcium (Ca), and strontium (Sr), and wherein the lanthanoid metal is selected from the group consisting of samarium (Sm), Rhodium (Rh) and Ruthenium (Ru);
B is a Group IV transition metal of the periodic table of the elements; and
X is an anion, and the anion is characterized by having a valence of minus 2 (−2); and wherein in the composition, the barium comprises from about 1 mole to about 2 moles, the Group IV metal comprises about 1 mole, and the lanthanoid comprises from about 0.1 mole to about 1.0 moles.

14. The composition as described in claim 13, wherein the Group IV metal is titanium.

15. The composition as described in claim 13, wherein the lanthanoid is samarium (Sm).

16. The composition as described in claim 16, wherein in the composition the barium, the titanium and the samarium are in a molar ratio of about 3:2:1 (Ba:Ti:Sm), respectively.

17. The composition as described in claim 15, wherein the composition is characterized by having a tolerance factor (“t”) of from about 0.8 to about 1.1, wherein t is defined by the equation:
t=(rₐ+rₒ)/(√2 (r_{b}+rₒ))
wherein in the crystal structure of the composition, rₐ and r_{b} are the ionic radii of cation species A and B, respectively, and rₒ is the ionic radius of the anion species.

18. The composition as described in claim 16, further comprising a support agent.

19. The composition as described in claim 18, wherein the support agent is selected from the group consisting of silicon dioxide (SiO₂), aluminum oxide (Al₂O₃), a mixture of silicon dioxide (SiO₂) and aluminum oxide (Al₂O₃), zeolites, magnesium oxide (MgO), barium oxide (BaO), calcium oxide (CaO), magnesium aluminum oxide (MgAl₂O₄), magnesium chromium dioxide (MgCr₂O₄), zirconium chromate (ZrCrO₄), zirconium aluminum oxide (ZrAl₂O₄), alpha-aluminum oxide (α-Al₂O₃), titanium oxide (TiO₂), cerium oxide (CeO₂) and zirconium oxide (ZrO₂).

20. The composition as described in claim 13, the composition prepared by a method comprising the steps of:
preparing a solution comprising:
the alkaline earth metal dissolved in an organic acid;
the Group IV transition metal dissolved in the organic acid;
the lanthanoid metal dissolved in the organic acid;
forming a combined solution comprising the dissolved alkaline earth metal, the dissolved Group IV transition metal, and the dissolved lanthanoid;
heating the solution for a time period sufficient to remove excess organic acid and form a gel;
drying the gel to form a powder;
heating the powder at increasing temperatures in a specified temperature profile; and
calcining the heated powder to form the perovskite composition.

21. A method of converting paraffinic hydrocarbons to alkenes, comprising the steps of:
employing a perovskite composition, the composition having the general formula of BaSmTiO₃, wherein in the composition, barium comprises from about 1 mole to about 2 moles, samarium comprises from about 0.1 mole to about 1.0 moles, and titanium comprises about 1 mole;
heating a reactor which contains the composition to a temperature ranging from about 400° C. to about 500° C. using a gas and a means for heating;
supplying a feed gas to the heated reactor, the feed gas comprising one or more paraffinic hydrocarbons, a quantity of oxygen, and a quantity of nitrogen, and wherein heat derived from converting the feed gas paraffinic hydrocarbon to alkenes heats the reactor to a temperature ranging from about 650° C. to about 1000° C., under conditions sufficient to convert the paraffinic hydrocarbon into one or more alkenes; and
collecting the gasses exiting the reactor, wherein the alkenes are present in the collected gasses.

22. The method as described in claim 21, wherein the paraffinic hydrocarbon is selected from the group consisting of methane, ethane, propane, butane, pentane, hexane, higher molecular weight alkanes (alkanes comprising greater than six carbon atoms) and naphtha.

23. The method as described in claim 21, further comprising the step of enriching the feed gas with an additional quantity of oxygen, and wherein the additional quantity of oxygen is steam.

24. The method as described in claim 21, wherein the alkenes are organic compounds comprising two or more carbon units (“C₂₊ compounds”), and the yield of C₂₊ compounds is in excess of 20%.

25. The method as described in claim 22, wherein the conversion of paraffinic hydrocarbons to alkenes is characterized by a selectivity of ethylene (“C₂H₄ selectivity”), and the C₂H₄ selectivity ranges from about 35% to about 80%.

26. The method as described in claim 22, wherein the alkene is ethylene.

27. The method as described in claim 21, wherein the reactor further comprises a plurality of means for heating, and the means for heating are distributed along the length of the reactor.

28. The method as described in claim 21, wherein the temperature ranges from about 750° C. to about 950° C.

29. The method as described in claim 28, wherein the temperature ranges from about 780° C. to about 850° C.

30. The method as described in claim 21, further comprising the step of adding a source of chlorine to the feed gas.

31. The method as described in claim 30, wherein the source of chlorine is chosen from the group consisting of carbon tetrachloride (CCl₄), chloroform (CHCl₃), chlorine gas (Cl₂), methane chloride (CH₃Cl), ethane chloride(C₂H₅Cl), methylene chloride, ethylene chloride, vinyl chloride, stannous chloride (SnCl₂) or other organic or inorganic chlorides and/or hydrochloric acid (HCl).

32. The method as described in claim 31, wherein the source of chlorine is chosen from the group consisting of carbon tetrachloride (CCl₄), chloroform (CHCl₃), and chlorine gas (Cl₂).

33. The method as described in claim 21, further comprising the step of adding a neutralizing agent to the feed gas.

34. The method as described in claim 33, wherein the neutralizing agent is selected from the group consisting of ammonia (NH₃), ammonium hydroxide (NH₄OH), methyl amine, dimethyl amine, trimethyl amine, ethyl amine, diethyl amine, triethyl amine and dimethyl ethyl amine.

34. The method of claim 33, wherein the neutralizing agent is selected from the group consisting of ammonia and ammonium hydroxide.

35. The method as described in claim 22, wherein the feed gas is heated to a temperature that is about the temperature of the reactor prior to adding the feed gas to the reactor.

36. The method as described in claim 22, further comprising the steps of separating the alkenes from the exit gasses to form a residual exit gas, and adding the residual exit gas back to the feed gas mixture.

37. The method as described in claim 21, wherein the perovskite composition is characterized by having a tolerance factor (“t”) ranging from about 0.8 to about 1.1, wherein t is defined by the equation:
t=(rₐ+rₒ)/(√2 (r_{b}+rₒ))
wherein in the crystal structure of the composition, rₐ and r_{b} are the ionic radii of cation species A and B, respectively, and rₒ is the ionic radius of the anion species.

38. The method as described in claim 27, wherein in the reactor, the perovskite composition is deposited between a layer of quartz.

39 The method as described in claim 38, wherein the reactor is lined with an inert liner.

40 A process for converting paraffinic hydrocarbons to alkenes in the presence of a catalyst composition prepared by the method of claim 1.
